(19) United States
(12) Patent Application Publication
(10) Pub. No.: US 2020/0277611 A1
(43) Pub. Date: Sep. 3, 2020

(54) PRESERVATION OF THE NEUROMUSCULAR JUNCTION AFTER TRAUMATIC NERVE INJURY

(71) Applicant: THE REGENTS OF THE UNIVERSITY OF CALIFORNIA, Oakland, CA (US)

(72) Inventor: Ranjan Gupta, Irvine, CA (US)

(21) Appl. No.: 16/871,574

(22) Filed: May 11, 2020

Related U.S. Application Data
(63) Continuation-in-part of application No. 16/700,640, filed on Dec. 2, 2019.
Application No. 16/700,640 is a continuation of application No. 14/133,414, filed on Dec. 18, 2013, now abandoned.
(60) Provisional application No. 61/738,912, filed on Dec. 18, 2012.

(51) Int. Cl. C12N 15/113; A61K 31/165; A61K 31/35; A61K 31/365; A61K 31/506; A61K 31/65; A61K 38/17; C07K 16/18; A61K 31/713; A61K 31/18; A61K 31/192; A61K 31/609

(52) U.S. Cl. CPC C12N 15/1137; A61K 31/165; A61K 31/35; A61K 31/365; A61K 31/506; A61K 31/609; A61K 38/1709; C07K 16/18; A61K 31/713; A61K 31/18; A61K 31/192; A61K 31/65

(57) ABSTRACT

Methods for treating nerve damage in a muscle, e.g., denervated muscle tissue (e.g., muscle damaged from traumatic injury), in a patient in need thereof featuring performing a pre-operative muscle biopsy on the denervated muscle tissue; making visible motor end plates (MEPs) in neuromuscular junctions (NMJs) in the biopsy; and performing a nerve transfer (e.g., partial radial nerve to axillary transfer) if (i) the MEPs shown in the biopsy persist and (ii) the MEPs shown in the biopsy retain their structures and exhibit certain morphometric properties. The nerve transfer helps regain neuromuscular function of the denervated muscle tissue. The biopsy may feature the use of two-photon microscopy. In certain embodiments, the method is performed at least 6 months after injury to the patient.

### CROSS-REFERENCES TO RELATED APPLICATIONS

This application is a continuation-in-part and claims benefit of U.S. patent application Ser. No. 16/700,640, filed Dec. 2, 2019, which is a continuation and claims benefit of U.S. patent application Ser. No. 14/133,414, filed Dec. 18, 2013, which is a non-provisional and claims benefit of U.S. Provisional Patent Application No. 61/738,912 filed Dec. 18, 2012, the specifications of which is/are incorporated herein in their entirety by reference.

### BACKGROUND OF THE INVENTION

### Field of the Invention

The present invention relates to methods, systems, and compositions for treating nerve injury, including methods for formulating diagnostic criteria to identify patients who may benefit from surgery.

### Background Art

Although the peripheral nervous system has the capacity for regeneration following injury, functional recovery after neural repair in adult humans remains limited. Despite surgical repair, there often still remains a poor outcome where the patient experiences only limited functional motor recovery. Some of the issues that may be associated with limited peripheral nerve regeneration include a lack of good scaffolding for regeneration, glial scar formation, poor peripheral support, and imprecise connections resulting in lack of coordination.

The current clinical decision tree for traumatic nerve injury in 2019 is: (1) If there is complete loss of function, and nerve transection is confirmed, consider surgery for re-apposition or bridging if the defect is large; (2) If there is complete loss of function, but nerve transection is not confirmed, consider watchful waiting to determine if there is spontaneous recovery; and (3) If there is no spontaneous recovery by six months, primary surgical nerve repair is considered futile, and nerve transfer can be considered to restore some useful movement.

The current clinical dogma that primary nerve repair after six months is futile assumes that denervation of muscle leads to degeneration of the MEP, the specialized postsynaptic region of the muscle fiber. Degeneration of the MEP impairs functional MEP reinnervation, despite subsequent axon regeneration. The evidence to support this clinical decision is based off anecdotal clinical observations and data from a few animal studies. The present invention suggests a different response to nerve injury in humans, distinct from small animal models. Specifically, in contrast to rodents, though they degenerate, denervated MEPs persist in humans for years (e.g. 10 years). Accordingly, mechanistic studies in rodents may be of limited value for defining the responses of MEPs to nerve degeneration in humans. Moreover, initial results from delayed nerve transfer surgery reveal that preserved MEPs in chronically denervated human muscle predict favorable outcome. This has profound implications for the clinical decision tree, opening treatment opportunities for chronic injuries that were previously not considered.

### BRIEF SUMMARY OF THE INVENTION

Without wishing to limit the present invention to any theory or mechanism, it is believed that morphological characteristics unique to a persistent motor endplate (MEP) as well as the presence of specific surrogate markers are associated with functional recovery following nerve repair surgery. Ultimately, this will guide prognosis after nerve injury as well as appropriateness of surgical intervention.

The present invention provides methods for determining the potential for neuromuscular recovery. The present invention also provides methods of treatment. For example, the present invention features methods for treating nerve damage in a muscle, e.g., denervated muscle tissue (e.g., muscle damaged from traumatic injury), in a patient in need thereof. In certain embodiments, the method comprises performing a pre-operative muscle biopsy (e.g., two-photon microscopy) on the denervated muscle tissue; making visible motor end plates (MEPs) in neuromuscular junctions (NMJs) in the biopsy; and performing a nerve transfer (e.g., partial radial nerve to axillary transfer) if (i) the MEPs shown in the biopsy persist and (ii) the MEPs shown in the biopsy retain their structures and exhibit certain morphometric properties. The nerve transfer helps regain neuromuscular function of the denervated muscle tissue

In certain embodiments, the method further comprises determining an innervation status of the MEPs in the biopsy. In certain embodiments, the method further comprises determining viability of the MEPs.

In certain embodiments, the method is performed at least 6 months after injury to the patient.

In certain embodiments, the method further comprises detecting at least one surrogate marker of reinnervation-competent muscle. In certain embodiments, the method further comprises administering a therapeutic agent to the denervated muscle tissue.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent application contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1A shows confocal images of human NMJs in an innervated deltoid. Red for α-bungarotoxin, green for neurofilament and synaptophysin, blue for DAPI. Scale bars=50 μm (20×)

FIG. 1B shows confocal images of human NMJs in a 5 month denervated first dorsal interossei. Red for α-bungarotoxin, green for neurofilament and synaptophysin, blue for DAPI. Scale bars=50 μm (20×)

FIG. 1C shows confocal images of human NMJs in 4 month denervated biceps. Red for α-bungarotoxin, green for neurofilament and synaptophysin, blue for DAPI. Scale bars=50 μm (20×)

FIG. 1D shows confocal images of human NMJs in 1 year denervated biceps. Red for α-bungarotoxin, green for neurofilament and synaptophysin, blue for DAPI. Scale bars=50 μm (20×)

FIG. 2A and FIG. 2B show 2-photon microscopy of human NMJs in innervated deltoid muscle. Red for α-bungarotoxin, green for neurofilament and synaptophysin. Scale bars=50 μm (20×)

FIG. 2C and FIG. 2D show 2-photon microscopy of human NMJs in biceps muscle 4 months after denervation due to traumatic peripheral nerve injury. Red for α-bungarotoxin, green for neurofilament and synaptophysin. Scale bars=50 μm (20×)

FIG. 3A and FIG. 3B show confocal images of human NMJs from biceps muscle one year after traumatic brachial plexus injury. FIG. 3A shows NMJ at low magnification. FIG. 3B shows NMJ at higher magnification. Red for alpha-bungarotoxin, blue for DAPI, green for neurofilament and synaptophysin.

FIG. 4A, FIG. 4B, FIG. 4C, FIG. 4D, and FIG. 4E show Hematoxylin & eosin staining of cross-sectional deltoid muscle fibers. Scale bars=100 μm (10×)

FIG. 4F, FIG. 4G, FIG. 4H, FIG. 4I, FIG. 4J show two-photon excitation microscopy of human NMJs. Red for α-bungarotoxin, green for neurofilament and synaptophysin. Scale bars=50 μm (20×)

FIGS. 5A-5C show the temporal profile of morphometric quantification of MEPs from human deltoids. FIG. 5A shows MEP volume, FIG. 5B shows MEP surface area and FIG. 5C shows MEP surface area to volume ratio. “D” denotes subject number. Months denervated noted in parentheses.

FIG. 6A, FIG. 6B, and FIG. 6C shows deltoid muscle biopsies stained for hematoxylin and eosin (scale bar=100 μm)

FIG. 6D, FIG. 6E, and FIG. 6F shows deltoid muscle biopsies stained for neuromuscular junction. α-bungarotoxin in red, neurofilament and synaptophysin in green. White arrowheads denote direct contact of pre-synaptic and post-synaptic elements.

FIG. 7A shows quantification of motor end plate surface area. Each data point represents one motor endplate from a single biopsy. Data are presented as mean±SEM.

FIG. 7B shows quantification of motor end plate volume. Each data point represents one motor endplate from a single biopsy. Data are presented as mean±SEM

FIG. 8 shows representative gross muscle appearance, H&E, Masson's trichrome, Oil Red 0, and MEP visualization with immunohistochemistry of human deltoid muscle 6 years after traumatic axillary nerve injury.

FIG. 9A shows a vervet monkey hand.

FIG. 9B shows Thenar muscle.

FIG. 9C shows MEPs of contralateral thenar control muscle.

FIG. 9D shows MEPs of a nerve injured muscle (at 9 months).

FIG. 10A, FIG. 10B, FIG. 10C, and FIG. 10D show Hematoxylin & eosin staining of cross-sectional deltoid muscle fibers.

FIG. 10E, FIG. 10F, FIG. 10G, and FIG. 10H show two-photon excitation microscopy of human NMJs. Red for α-bungarotoxin, green for neurofilament and synaptophysin.

FIG. 10I, FIG. 10J, FIG. 10K, and FIG. 10L show clinical Images showing prominent deltoid atrophy in patients with axillary nerve injury.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application. One skilled in the art will recognize many methods and materials similar or equivalent to those described herein, which could be used in the practice of the present invention. The present invention is in no way limited to the methods and materials described.

Without wishing to limit the present invention to any theory or mechanism, it is believed that morphological characteristics unique to a persistent motor endplate (MEP) as well as the presence of specific surrogate markers are associated with functional recovery following nerve repair surgery. Ultimately, this will guide prognosis after nerve injury as well as appropriateness of surgical intervention.

### Human Motor Endplate Remodeling in Traumatic Denervation

Patients with traumatic brachial plexus injuries (BPI) have particularly poor outcomes with limited functional recovery, even after optimal surgical management. As improvements in recovery have plateaued secondary to surgical manipulations alone, adjuvant cellular and molecular therapeutic regimens are required. Yet, the appropriate time to intervene can only be determined if there is a true understanding of the process of nerve and muscular degeneration secondary to a traumatic nerve injury. While animal models have shed light on molecular changes to the muscle and motor endplate post-injury, the time course of degeneration in animal models is unlikely to be the same as in the human condition, and thus cannot provide precise information that would help inform surgical intervention and the timing for adjuvant therapy.

Clinical studies in this area have focused on advances in surgical treatment including primary repair, reconstruction using autograft, allograft, or nerve conduits, and, more recently, nerve transfers. However, these efforts only partially restore function to the affected limb, may result in donor site morbidity, and often yield unpredictable outcomes. One reason for these unpredictable results may be post-synaptic changes at the neuromuscular junction (NMJ) resulting in irreversible motor endplate degradation. The NMJ is the interface between the peripheral nervous system and the muscles it innervates. It consists of a presynaptic nerve terminal, a motor endplate, and terminal Schwann cells. Following axonotmesis or neurotmesis, Wallerian degeneration and axonal regeneration may result in reconstitution of the presynaptic nerve terminal under appropriate conditions. However, if the regenerating axons fail to reach the target muscle prior to degradation of the motor endplate, signal transduction across the NMJ cannot proceed, effectively resulting in permanent denervation of the target muscle.

Animal studies have demonstrated that the architectural arrangement of acetylcholine receptors (AChRs) at the post-synaptic motor endplate is critical for effective signal transduction at the neuromuscular junction. Long-term denervation results in declustering and dispersion of AChRs, leading to disassembly of the motor endplates. Destruction of normal motor endplate architecture results in failure of synaptic transmission at the NMJ and severs communication between the nervous and musculoskeletal systems. This phenomenon effectively limits the therapeutic window for operative intervention in patients with axonotmesis or neurotmesis, with resulting atrophy of the target muscles.

One of the greatest challenges facing translational research in this area are species-specific differences between human and murine NMJs. Recent studies have demonstrated that human NMJs are morphologically distinct and molecularly divergent from murine NMJs. Furthermore, contrary to murine findings which have suggested that NMJ degeneration increases with age, human NMJs have been shown to remain stable across the adult lifespan.

The current understanding of neurologic injury and regenerative outcomes in humans is based solely on clinical observations. Outcomes have been found to depend on a number of factors including patient age, level of injury, gap size (in the case of transection), patient comorbidities, smoking status, associated injuries, and timing to surgery. Of these, the only factor that can be modified by the surgeon is timing to surgery. Historically, studies suggest that even in distal lesions, surgical interventions that take place more than six months after injury rarely result in meaningful recovery. Although this may suggest that post-synaptic motor endplate degradation is approximately complete and irreversible by this time point, no studies have histologically examined or described the temporal profile of human motor endplate degradation after acute nerve injury. While animal studies and clinical observations have historically served to guide surgical decision making, appropriate timing of surgical intervention in humans can only be conclusively determined with a more thorough understanding of the mechanisms underlying motor endplate degeneration following acute nerve injury in humans. The present invention helps to provide evidence to aid surgeons in determining appropriate timing for operative intervention in these injuries.

Materials and Methods: IRB approval was obtained to perform biopsies from denervated muscles in patients with nerve injuries ranging from complete pre-ganglionic C5-T1 BPI to less severe traumatic injuries such as isolated axillary nerve transections. Prior to performing any surgical intervention, electromyography was performed by a board certified neuromuscular trained neurologist to confirm the absence of axillary nerve action potentials, along with presence of fibrillation potentials and positive sharp waves in all patients. Muscle biopsies were obtained from 13 patients beginning in 2015 with a total of 11 deltoids, 1 bicep, and 2 first dorsal interossei. There were 12 male and 1 female patient with an age range of 19 to 46. The time from injury to muscle biopsy ranged from 7 days to 6 years. The timing of the muscle biopsy was based on the time of presentation to the operating surgeon along with the clinical decision-making to provide the patient with the best therapeutic option currently available. As such, muscle samples from multiple time points after injury were analyzed, along with control specimens from innervated muscles to create a temporal sequence of events for human motor endplate degradation following traumatic nerve injury.

Specimens from the operating room were cryoprotected in Tissue Tek OCT mounting medium (Torrance, Calif.) and snap-frozen using liquid nitrogen. 20-micrometer cross-sections were generated at −20° C. using a microtome. Sections were then stained with hematoxylin and eosin (H&E) to evaluate the overall tissue composition and structure. Images were captured using an inverted microscope (IX71, Olympus).

A subset of specimens from the operating room was immediately snap-frozen, processed for immunohistochemistry, and visualized via confocal and two-photon microscopy. Following overnight fixation, specimens were incubated in recombinant mouse anti-human synaptophysin ( 1/250, Dako), and purified mouse anti-human neurofilament ( 1/300, Covance), to label presynaptic vesicles and axons, respectively. After rinsing, specimens were then incubated in secondary antibodies conjugated to donkey anti-mouse Alexa Fluor® 488 ( 1/300, Thermo Fisher) and alpha-bungarotoxin, Alexa Fluor® 594 conjugate ( 1/1000, Thermo Fisher), to directly label motor endplates.

Tissue samples were visualized via two-photon excitation microscopy due to its primary advantage of superior optical sectioning in three-dimensional imaging of thick specimens. The deeper penetration of two-photon excitation compared to standard confocal imaging allows for assessment of the spatial arrangement and morphometric properties of NMJs. Two-photon excitation was achieved by using an 810 nm laser to excite both fluorophores simultaneously. Images were acquired with a custom microscope system by Intelligent Imaging Innovations™ using a 20×/0.8 water immersion objective lens. Images obtained via 2-photon excitation microscopy were used to create three-dimensional reconstructions with Volocity imaging software (Perkin Elmer) to allow for precise quantification of morphometric properties of NMJs.

Results: All muscle biopsies were taken from patients with a definitive, clearly identifiable date of injury and were obtained during a clinically indicated standard of care surgical intervention, with informed consent obtained from all patients. As such, it was possible to obtain muscle biopsies from the same surgical incision used for these procedures, which ranged from a partial radial nerve to axillary nerve transfer to an Oberlin transfer. After analysis of the 11 deltoids, 1 bicep, and 2 first dorsal interossei, there was no detection of any muscle-specific pattern of degeneration; rather, there appeared to be a time dependent pattern of degeneration. Gross atrophy of denervated deltoid muscle was clinically apparent. Biopsy of these muscles showed marked histological changes, including shrinkage of muscle fibers and peri-fascicular fat accumulation.

Denervated first dorsal interossei, bicep, and deltoid muscle samples showed distinct differences from innervated muscles of control specimens, including fragmentation and dispersion of acetylcholine receptors, as well as a trend towards plaque endplate morphology. Endplate morphology has previously been characterized as a spectrum ranging from mature pretzel endplates to immature plaque endplates, according to their distinct topographic features at the postsynaptic membrane. Mature pretzel endplates are defined by their web-like patterning and multiple perforations, while immature plaque endplates are defined by their smaller size and lack of perforations. This morphometric dichotomy, as the motor endplate transitions from plaque to pretzel, is a hallmark of neuromuscular junction development. Interestingly, the phenomenon of denervation seems to cause the mature pretzel morphology to regress back to the plaque morphology of the early embryonic developmental state.

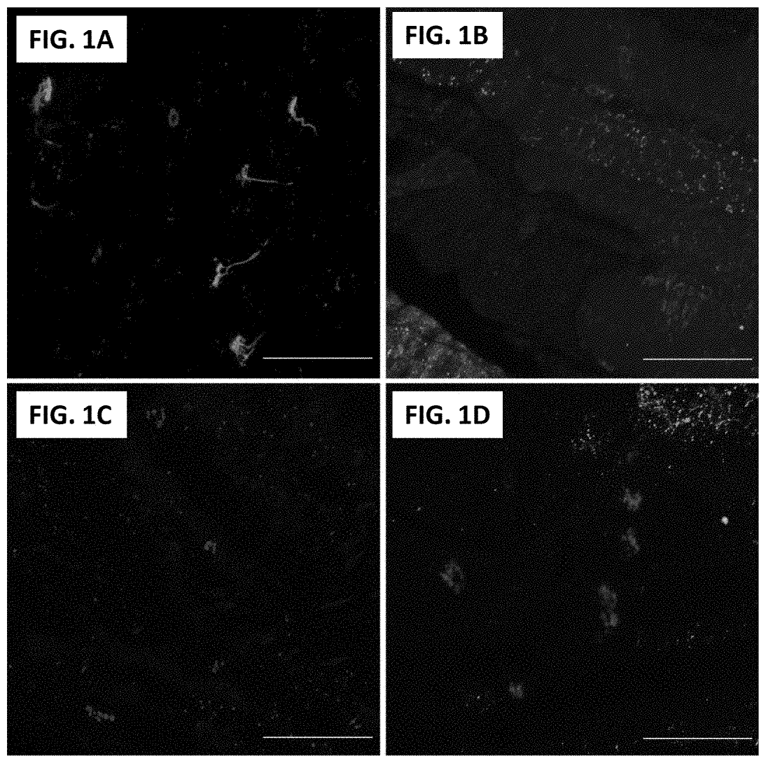

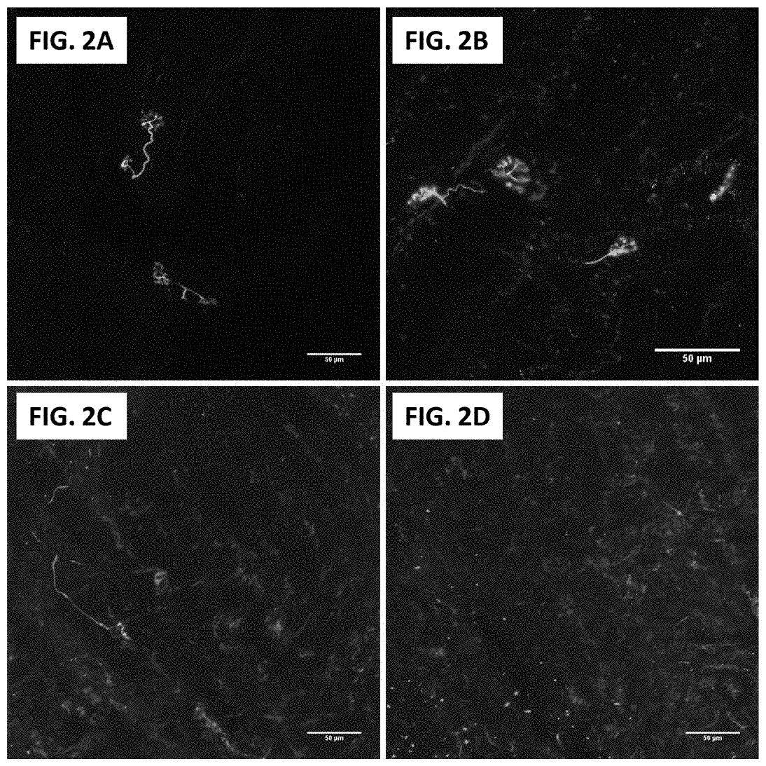

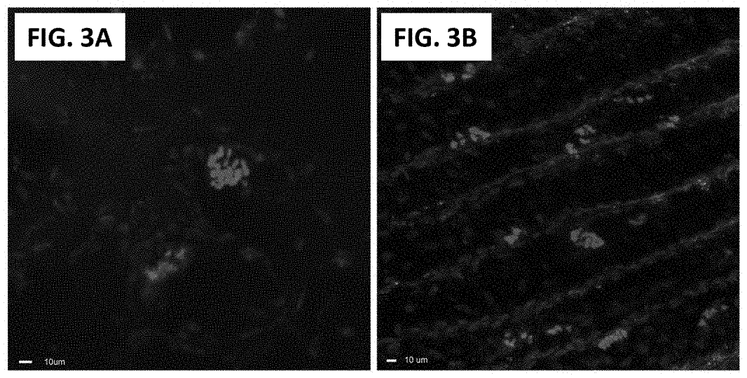

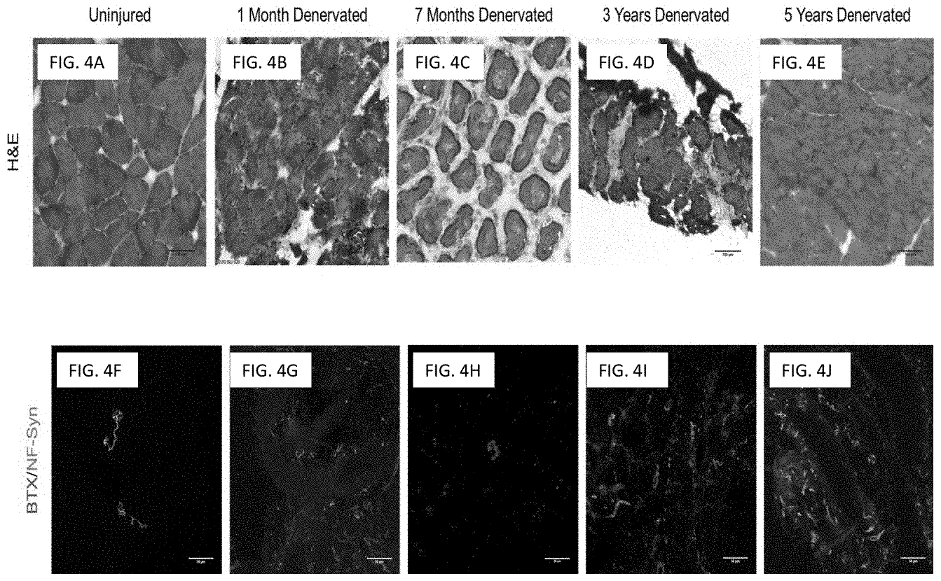

Morphologic comparison of denervated first dorsal interosseous, bicep, and deltoid muscles showed signs of temporal degeneration. NMJs from recently denervated muscles demonstrated well-preserved circular morphology, with acetylcholine receptors arranged in distinct folding patterns (FIG. 1B, FIG. 1C, FIG. 2C, FIG. 2D, FIG. 4G, & FIG. 4H). By one year, NMJs began demonstrating greater fragmentation (FIG. 1D & FIG. 3). Moreover, synaptic gutters started to fade, and asymmetry in acetylcholine receptor distribution was noted (FIG. 1D & FIG. 3). Interestingly, even after one year of denervation, morphologically normal NMJs persisted. Although images using 2-photon microscopy revealed a decrease in NMJ volume as seen in 3D reconstruction, as well as loss of mature AChR morphology and trend towards plaque endplate morphology, motor endplates did not demonstrate complete degeneration and disintegration (FIG. 4I & FIG. J).

Overall, comparison of denervated muscles showed signs of temporal degeneration. NMJs from acutely-denervated muscles demonstrated well preserved circular morphology with definite acetylcholine receptors arranged in distinct folding patterns while NMJs from more chronically-denervated muscles revealed a trend towards plaque endplate morphology. Remarkably, innervated morphologically preserved NMJs persisted in muscles that had been denervated for greater than 5 years (see FIG. 4E, FIG. 4J).

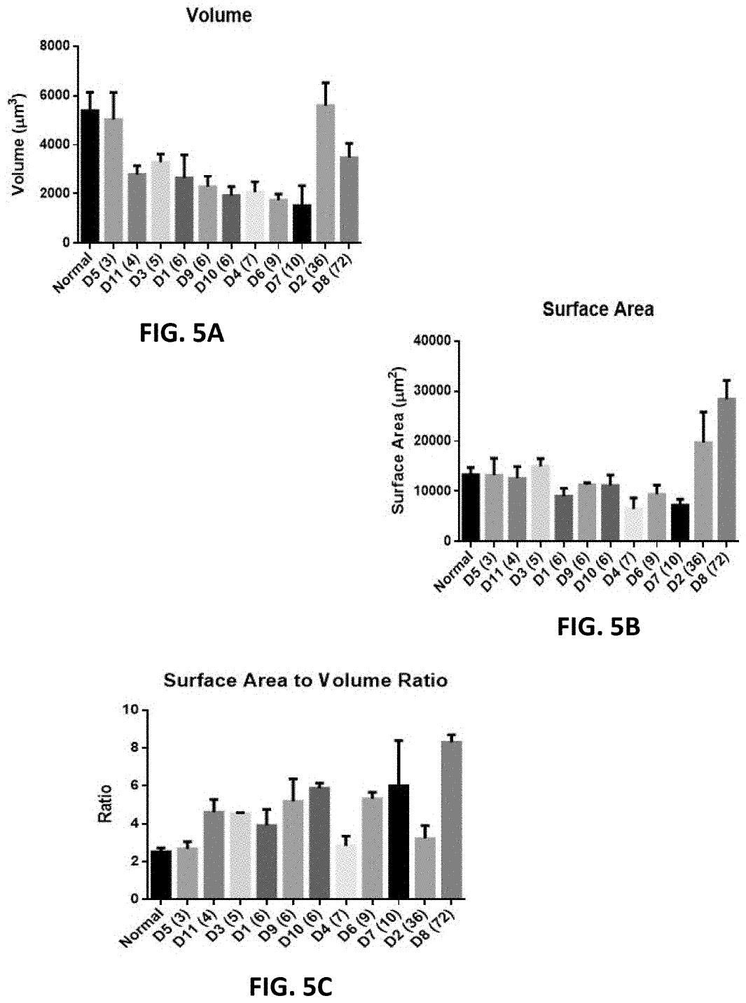

Referring to FIG. 5, morphometric quantification of MEPs from denervated deltoids compared to normal deltoid revealed a decrease in MEP volume at the 3 months to 10 months post-denervation timepoints with increase in MEP volume close to normal in the 36 months and 72 months post-denervation timepoints. MEP surface area revealed relative stability at the 3 months to 10 months post-denervation timepoints compared to normal deltoid while deltoids denervated for 36 months and 72 months revealed an increase in MEP surface area relative to normal deltoid.

Discussion: In this study we describe a novel method for assessing the functional potential of denervated human muscle tissue, as well as characterize the temporal profile of motor endplate degeneration after traumatic peripheral nerve injury. Surprisingly, human NMJs persist and retain their architecture even after the six-month window of opportunity for meaningful functional recovery has elapsed. These findings contradict the expectation that motor endplates would have dispersed by the end of the critical six-month window after injury.

Rodent models have shown that the release of agrin by motor neurons is critical to the aggregation of acetylcholine receptors (AChRs) at the motor endplate, and that damage to the NMJ results in molecular changes to the motor endplate, including the dispersion of AChRs. It has been previously demonstrated that long-term denervation is characterized by alterations in the motor endplate morphology from a mature pretzel (perforated and containing membranous infoldings) appearance to an immature plaque (diminished size and increased density), as well as an intermediate morphology in between the two. This switch to a plaque-like morphology is directly correlated with the critical time window beyond which functional recovery by reinnervation is severely limited, due to degeneration of the NMJ. An understanding of this degradation process is critical and applies not only to the treatment of traumatic peripheral nerve injuries, but also to progressive neurodegenerative diseases. For example, denervation without motor neuron loss has also been implicated in the early stages of amyotrophic lateral sclerosis in murine models as well as in humans.

The observed persistence of human MEPs after prolonged denervation may be explained by a few possible phenomena. These persistent MEPs may arise from newly formed MEPs associated with new regenerating muscle fibers formed after denervation. Recent findings also suggest a likely role of sympathetic innervation at the MEP in the regulation of AchR stability and maintenance which may explain the persistence of human MEPs despite long-term motor denervation. For instance, sympathetic ablation experiments via surgical or chemical sympathectomy have implicated sympathetic-dependent regulation of the levels of postsynaptic membrane AchRs through AChR recycling and degradation via sympathetic control of two main pathways, PKA/cAMP and Gαi2-Hdac4-Myogenin-MuRF1 pathways. Moreover, studies of chemical sympathectomy of mouse tibialis anterior muscles resulted in significant electrophysiological and morphological deficits of the MEPs, but both phenotypes were rescued when treated with a sympathomimetic drug, suggesting a critical role of sympathetic innervation in the homeostatic maintenance of MEPs.

In order to determine whether adjuvant therapies which have shown efficacy in mice warrant clinical trials in humans, it is necessary to first understand how human NMJ degeneration differs in both process and timing. Whereas murine models show significant functional deficits when reinnervation occurs beyond two months following injury, clinical observations suggest that this does not translate to optimal timing of surgical intervention in humans. One suggested surgical solution for treatment of axillary nerve injuries is a partial radial nerve to axillary transfer. However, this procedure has variable results and is not currently widely accepted. If human NMJs persist and retain their structures even after the previously postulated 6-month therapeutic window for operative intervention, then a partial radial nerve to axillary transfer may indeed be indicated. The decision to undertake a nerve transfer is currently made clinically, with little guidance from objective data. The results suggest that a pre-operative muscle biopsy may offer additional data to aid in the decision-making process by providing direct visualization of the neuromuscular junction, including its innervation status and morphometric properties. This data can assist in predicting which nerve transfers are likely to be successful.

Examination of the Human Motor Endplate after Brachial Plexus Injury with Two-Photon Microscopy

After brachial plexus injuries (BPI), some patients experience spontaneous recovery, whereas others require surgical intervention to improve functional outcomes. Formulating diagnostic criteria to identify patients who may benefit from surgery is a high priority. MRI and ultrasound are both useful for identifying damaged nerves pre-operatively, but cannot predict regeneration potential, which depends on the viability of the motor end-plate (MEP) within targeted muscle fibers.

Without definitive diagnosis of nerve transection and with an inability to track viability of the neuromuscular junction (NMJ), many surgeons delay surgery to assess spontaneous recovery and avoid unnecessary or potentially detrimental procedures. As human nerves grow at a rate of ˜1 mm per day, it often requires months before clinical signs of regeneration are apparent. However, late surgical intervention risks irreversible degradation of the target end-organ, thus missing the critical window during which functional recovery is achievable.

To develop tools for predicting spontaneous neuromuscular recovery, a deeper understanding of the fate of human MEPs following denervation is crucial. The present invention describes an approach to visualize human NMJs in muscle biopsies.

Case Reports:

Two young, healthy males, ages 26 and 23 years, sustained similar gunshot wounds to the right upper extremity within minutes of each other during a mass casualty incident. Both patients had BPIs including complete motor and sensory loss in the right axillary nerve distribution without transection injury and required standard-of-care surgeries to address their bony injuries where the deltoid muscle was readily accessible. After receiving institutional review board (IRB) approval and obtaining informed consent, the right deltoid muscle was biopsied in these patients at 3 weeks and 5 months, respectively, allowing comparison with a control deltoid biopsy from a subject undergoing upper extremity surgery unrelated to a nerve injury.

Muscle Processing and Analysis:

Muscle samples were fixed in 4% paraformaldehyde, separated into longitudinal whole mounts, and immunostained with antibodies to NMJ components: 1) alpha-bungarotoxin (Alexa Fluor® 594 conjugate; 1/1000, Thermo Fisher) to label nicotinic acetylcholine receptors; 2) synaptophysin to label presynaptic vesicles (mouse anti-human synaptophysin; 1/250, Dako); 3) neurofilament (NF) to label axons (mouse anti-human NF: 1/300, Covance). Secondary antibodies were conjugated to donkey anti-mouse Alexa Fluor®-488 ( 1/300, Thermo Fisher). Two-photon images were acquired with a 3i system (Intelligent Imaging Innovations™) with 810 nm laser and Zeiss 20×/0.8 water immersion objective. Three-dimensional reconstructions were created (Volocity, Perkin Elmer). MEP surface area/volume were quantified using ImageJ with the 3D Object Counter plugin using the optical fractionator method. Hematoxylin & eosin (H&E) staining was used to visualize muscle fiber architecture in transverse cryosections of fresh frozen muscle.

Results: Electrodiagnostic studies were performed on Patients 1 and 2, demonstrating indicating deficits in the distribution of the right median, ulnar, radial, and axillary nerves in Patient 1, and in the right axillary and radial nerve distributions in Patient 2.

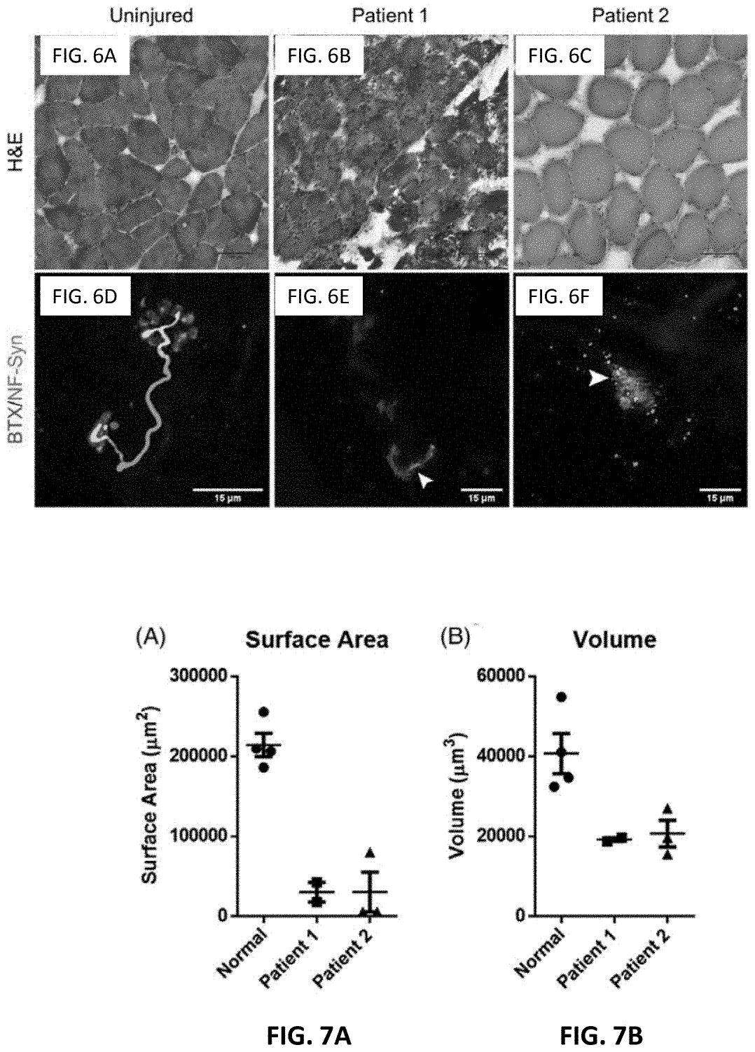

On H&E staining, Patient 1 demonstrated highly variable muscle fiber diameters with diffuse, dense cellular infiltrate throughout the specimen, consistent with early myofiber regeneration (see FIG. 6B). In contrast, Patient 2 demonstrated uniform fiber diameters, indicating normal muscle morphology (see FIG. 6C). Neither specimen exhibited changes typical of late stages of muscle injury (adipocyte infiltration or collagen deposition). Uninjured specimen is shown in FIG. 6A and FIG. 6D.

The deltoid biopsy from Patient 1 (3 weeks post-injury) showed extensive neurofilament debris scattered throughout the field compared to the control indicating active Wallerian degeneration (FIG. 6E). The biopsy from Patient 2 (5 months post-injury) also showed neuronal debris, consistent with late Wallerian degeneration. In both patients, synaptophysin signal was in contact with some, but not all MEPs (white arrowheads, FIG. 6E, FIG. 6F). MEPs of both patients were grossly intact but showed marked condensation with loss of infoldings compared to controls, an 86% reduction of surface area (FIG. 7A) and decreases in endplate volume of 53% and 49% (see FIG. 7B) for Patients 1 and 2, respectively.

Patient 1 started to regain both motor and sensory function in the distribution of the axillary nerve six months post-injury, and eventually regained function in all right upper extremity muscles. Patient 2 spontaneously regained deltoid muscle function (Medical research Council (MRC) grade 3) six months post-injury, eventually regaining MRC grade 5 deltoid strength over the next year along with full radial nerve function including independent digital extension after nerve transfers. This imaging data was not used to alter clinical management.

Discussion: A crucial decision in management of traumatic BPI is whether to perform surgical intervention. These two cases provide insights into how human nerves and MEPs respond to gunshot-induced BPI. The clinical course of both patients suggests that they sustained a reversible neurapraxia secondary to ballistic shock waves and subsequent soft tissue swelling. At the time of clinical presentation, electrodiagnosis and imaging could not distinguish between a pressure wave injury and irreversible axillary nerve damage.

There is animal data about the NMJ response to injury focused on terminal Schwann cells as well as molecular changes to the MEP, including the dispersion of AChRs. Long-term denervation is accompanied by devolution of the MEP morphology from a mature pretzel appearance (perforated with membranous infoldings) towards an immature plaque (diminished size/increased density). This transition to a plaque-like morphology is correlated with the critical time window beyond which reinnervation and functional recovery is severely limited. We used two-photon microscopy because it provides superior optical sectioning in three-dimensional imaging of thick human specimens compared to standard confocal imaging. The deeper penetration of two-photon excitation allows visualization and accurate quantification of morphometric parameters. We observed significant neurofilament and synaptophysin debris in Patient 1, suggesting active Wallerian degeneration, as expected 3 weeks after traumatic nerve injury. MEP morphometric changes were evident in both patients, including decreases in surface area and volume, with increased density, consistent with the change to a plaque-like phenotype seen in murine models of traumatic nerve injury. Taken together, these models show that these changes are reversible.

Understanding the nature and time course of changes in MEPs after nerve injury is critical to the development of an evidence-based decision process. However, these have not been studied in humans, and it is unknown whether the sequence of events and time course are accurately represented by animal models. Biopsy of brachial plexus nerve fascicles has been undertaken for diagnosis of neuropathologic states, but biopsy of denervated muscles and visualization of the MEP have not been done. Thus, our approach is a critical first step towards understanding these processes in humans. An understanding of the nature and time course of degeneration of NMJs is also important for progressive neurodegenerative diseases. Denervation without motor neuron loss has also been implicated in the early stages of amyotrophic lateral sclerosis in both murine models and humans. The late stages of chronic nerve compression have also been shown in animal models to resemble the sequelae of traumatic nerve injury.

### Example 1

The following is a non-limiting example of the present invention. It is to be understood that said example is not intended to limit the present invention in any way. Equivalents or substitutes are within the scope of the present invention.

The present invention discloses 1) assessing the nature and time course of human MEP degeneration in post-traumatic nerve injury; 2) identifying the morphometric characteristics of the MEPs and the surrogate molecular markers from a pre-operative muscle biopsy indicating receptivity to reinnervation; and 3) developing a model system within the CTSA framework for assessing outcomes from surgical interventions in order to generate evidence-based diagnostic guides.

The problem of peripheral nerve regeneration can be caused by the failure of axon regeneration back to appropriate targets. Adult axons regenerate at a peak rate of 1-3 millimeters per day. For proximal peripheral nerve lesions, axons must regenerate for tens of centimeters and cannot reach targets for months. The problem of peripheral nerve regeneration can also be cause by the failure of reinnervation of the target (muscle in the case of motor axons). The present invention discusses the failure of reinnervation, which is due in part to time-dependent changes in denervated muscle.

The connection between peripheral nerve and muscle occurs at the neuromuscular junction (NMJ), which is composed of the terminal branch of the motor axon, peri-synaptic Schwann cells, and the motor end plate (MEP), a specialized region of the muscle fiber containing acetylcholine receptors (AChRs). In rodent nerve injury, Wallerian degeneration of axons causes several end organ changes through mechanisms that are not fully understood. This culminates in the loss of the MEP, limiting the ability of regenerating axons to re-establish functional connections. Recently, Inventors have defined time-dependent decreases in MEP area and receptor density in rodents. Delaying these changes can enhance reinnervation. These animal data support that loss of the MEP is a key change leading to reinnervation failure and that functional reinnervation can be achieved with preservation of the MEP.

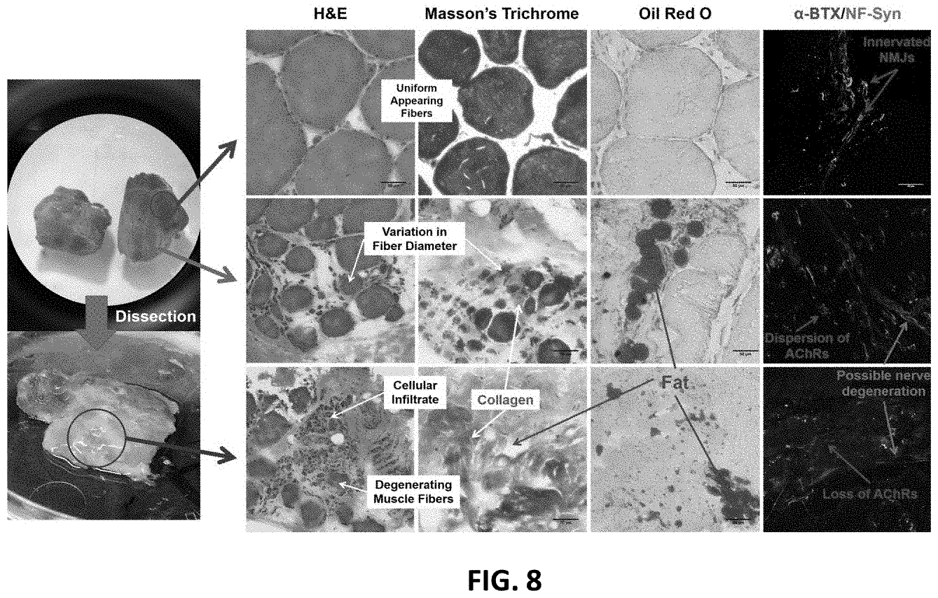

Although animal studies have revealed important biological mechanisms, species-specific differences in morphology and molecular composition of MEPs exist between humans and rodents. Preliminary data indicates species-specific differences in MEP degeneration secondary to nerve injury. Specifically, in contrast to rodents, where MEP degeneration is complete by 4 months, this data reveals that MEPs persist in denervated human muscles for years (FIG. 8).

Given that understanding biological mechanisms informs clinical decisions, the findings of human-specific processes compel further study of human specimens following naturally occurring traumatic injuries. Of importance are the factors underlying the preservation of human MEPs and would enable functional reinnervation at prolonged time intervals after injury. This information alone would fundamentally modify the current surgical decision tree.

Morphometric Analysis of Human MEPs from Patients with Traumatic Nerve Injury to Determine the Nature and Time Course of Human MEP Degeneration

Studies in humans are essential for developing evidence-based decision criteria and eventually new, effective therapies. A longitudinal cross-sectional study of MEP degeneration following injury for biopsy specimens from patient groups would define the timing and sequence of structural changes of human MEPs following nerve injury. A secondary goal is to obtain human-specific data on the morphological features associated with preservation of denervated MEPs.

Measures. Muscle biopsies will be taken intra-operatively from the denervated muscle; our current IRB protocol allows harvest of six grams of muscle. Biopsies will be processed for immunohistochemistry as well as hematoxylin and eosin (H&E) staining (0.5 grams for MEP morphometry and 0.2 grams for muscle fiber histology). Immunohistochemistry with visualization by two-photon excitation microscopy will be used to image MEPs of denervated muscle biopsies as well as innervated, control biopsies from routine orthopedic procedures. Two-photon microscopy will be used for optical sectioning and imaging to create three-dimensional reconstructions for precise quantification and analyses of morphometric properties. Two-photon imaging provides superior depth penetration and spatial resolution compared to standard confocal or fluorescent imaging. Optical stacks acquired with our custom microscope system by 3i Intelligent Imaging Innovations™ are analyzed with Volocity® Quantitation software, which allows re-construction and 3D rendering with correction of optical distortion by deconvolution and quantitative morphology of MEP surface area and volume. As control samples, biopsies from innervated muscles will also be analyzed for the same characteristics. This standardized method of quantifying surface area and volume can be used across sites to provide a pipeline for assessing endplate remodeling in a wide range of injury and disease models. Biopsies stained for H&E will also be used to define changes to the overall tissue composition and microstructure of denervated muscles. Degeneration and fat distribution changes to the muscle fibers will be quantified by scoring for evidence of degenerating fibers as well as intrafascicular and perifascicular fat per fascicle with the results reported as a percentage of total fascicles per sample.

Preliminary findings indicate that some surviving MEPs in functionally denervated human muscle are actually innervated, but the type of axon is unknown. These could be motor axons that have sprouted from nearby innervated muscles, sympathetic axons as described previously, or sensory axons. This will be explored by immunostaining biopsy specimens using antibodies for the three different axon types to determine whether the presence of a particular axon type at the MEPs correlates with overall preservation of MEPs. The presence of motor, sympathetic, or sensory axons at the MEP will be quantified by measuring presynaptic to postsynaptic occupancy. The occupancy of each axon type will be calculated as a percentage of total presynaptic area vs. postsynaptic area (pre/post*100).

The patient-specific data of MEP surface area and volume, axon-specific presynaptic to postsynaptic occupancy, and histological changes to denervated muscle fibers with functional outcomes following surgical intervention will be compared. To define the evolution of changes in MEPs, data from patients receiving surgery at different times post-injury will be compared.

Morphometric Analysis of Non-Human Primate (NHPs) MEPs after Surgically Created Complete C5-T1 Brachial Plexus Injury

Studies of MEP persistence after injury are from clinical situations where the physical exam, electromyography, muscle biopsy, and histology indicate functional nerve transection and muscle denervation. However, the actual site of the nerve injury cannot ethically be directly examined and histologically evaluated in humans. Clinical exams also cannot exclude the possibility of some axonal sparing which might contribute to the preservation of MEPs. Further, traumatic injuries in humans can damage different nerves leading to denervation of different muscles. Thus, it is important address these sources of variability through studies in a non-human primate (NHP) model with controlled nerve injuries. The present invention describes using vervet monkeys (Chlorocebus pygerythrus), taking muscle biopsies at defined time points post-injury.

Manipulations. NHPs will be housed and treated in compliance with NIH non-human primate guidelines. Complete pre-ganglionic C5-T1 transections will be performed in a controlled surgical procedure with critical gaps created to eliminate the possibility of axon sparing. Muscle biopsies will be harvested from denervated muscle at defined intervals, matching the time-course represented by the human samples. Serial biopsies from 5 vervet monkeys will be sampled at a monthly interval up to one-year post-injury creation.

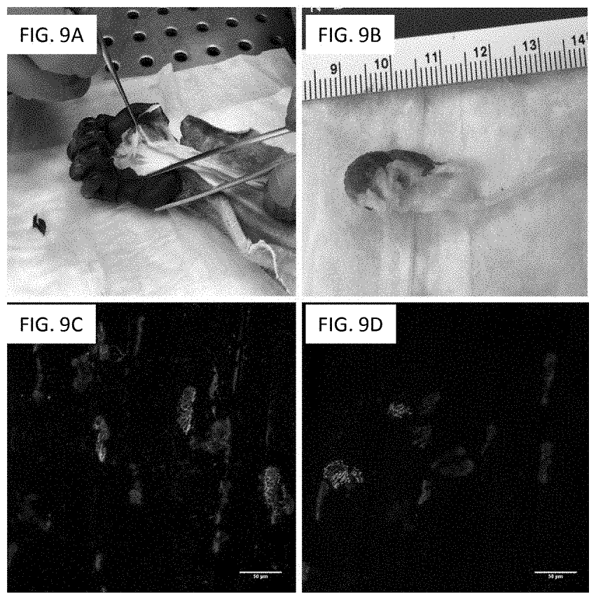

Measures. The muscle biopsy protocol used will be the same that is used for human samples. Muscle biopsies will be processed for immunohistochemistry and hematoxylin and eosin (H&E) staining; two-photon microscopy will be used for 3D reconstructions. As to be expected, the closeness in the phylogenetic tree between NHP and humans has allowed the use of the same methods to acquire human MEP images in the vervet monkey with minimal modifications required (FIG. 9).

Outcomes. The studies herein will provide novel data on the evolution of degenerative changes in human and NHP MEPs following nerve injury as well as the relationship between preservation of MEPs and functional outcomes following surgery. This data will provide a framework for data-driven decisions on the timing of surgical or other therapeutic interventions. It is envisioned that a regression analysis will identify temporal thresholds in both human and NHP data sets at which morphological outcomes of MEP area and volume decay to, for example, 50% or 20% of their maximum.

Identify the Morphometric Characteristics of the MEPs and the Surrogate Molecular Markers from a Pre-Operative Muscle Biopsy that Indicate Receptivity to Reinnervation

Problems. Current clinical dogma and observational studies dictate that surgical interventions more than 6 months after nerve injury rarely result in meaningful recovery. At the same time, surgical interventions are often postponed in hope that spontaneous recovery will occur. For example, with simple stretch injury (neuropraxia) there may be temporary loss of transmission that will recover without intervention. However, axonotmesis or neurotmesis injuries (in which the axons or the entire nerve are transected, respectively) are often clinically misdiagnosed as a simple neuropraxia. In this case, waiting for spontaneous recovery that will not occur leads to delays well beyond the 6-month time frame, ultimately precluding any success of surgical intervention to repair the nerve. In the absence of spontaneous recovery, nerve transfer offers the best opportunity for functional recovery. In this technique, a portion of a nearby functional nerve is transferred to the injured nerve to restore function. However, an important caveat is that nerve transfer can only work if the transferred nerve forms functional connections with denervated muscle. Thus again, muscle receptivity becomes a limiting factor. This may be why there are widely varying outcomes after nerve transfer surgery.

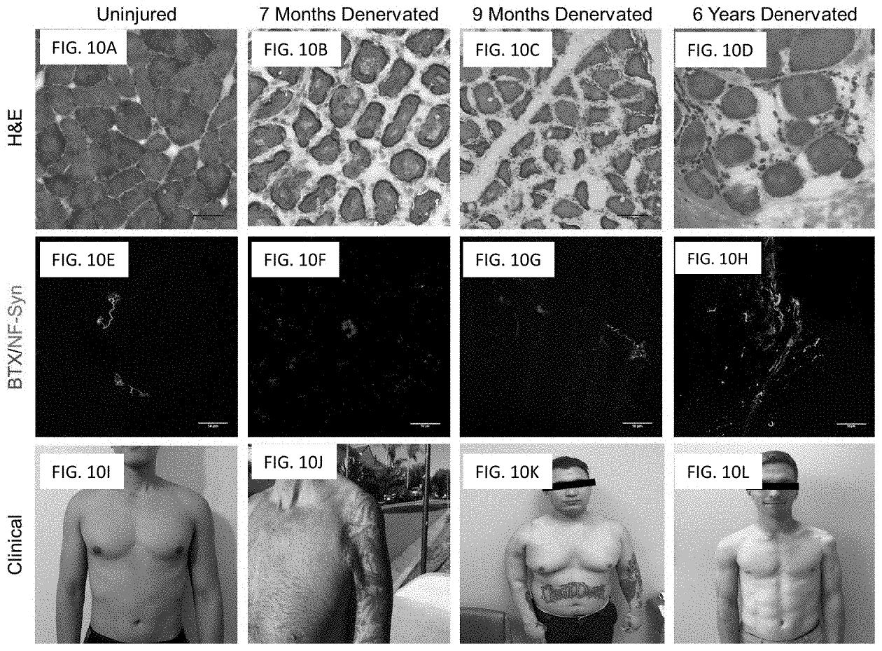

Solutions & Rationale. Preliminary data indicates persistence of MEPs beyond the six-month time point in some patients and that surgical repair in such individuals can be effective. Three patients who presented greater than 6-months post-injury, including one patient presenting 6-years post-injury, showed persistence of MEPs (FIG. 10). Markedly, all three patients demonstrated functional muscle recovery and return of normal muscle bulk on follow-up physical examination after a nerve transfer. Accordingly, muscle biopsy to detect surviving MEPs could provide clinical evidence that the critical window for surgical intervention and reinnervation is still open in select patients, regardless of the time post-injury. It is possible that a preoperative muscle biopsy to evaluate persistence of MEPs may provide an objective prognostic tool to aid in determination of surgical candidacy and in predicting post-operative outcomes.

Another objective, prognostic/diagnostic tool is human-specific surrogate molecular markers at the MEP, indicative of reinnervation-competent muscle. If reliable surrogate markers of reinnervation-competent muscle can be identified, then objective, clinical indications for reinnervation-competent human muscles can be developed to guide surgical decision-making based on a novel approach of molecular pre-screening. The present invention describes the use of a broad, unbiased data collection approach involving proteomic analysis of biopsy samples. Although the primary goal is to identify potential surrogate molecular markers of a reinnervation-competent muscle, the human-specific data will also directly generate testable hypotheses concerning the molecular mechanism of human-specific MEP degeneration.

Determine if the Persistence and/or Certain Morphometric Characteristics of MEPs on a Pre-Operative Muscle Biopsy Correlates with Improved Functional Recovery Following Nerve Transfers.

Patient population. The patient population will be patients with functionally complete injuries that have not experienced recovery who are undergoing nerve transfer surgery ranging from a partial radial nerve to axillary nerve transfer to an Oberlin transfer.

Measures. At the time of their surgical procedure, a biopsy will be taken and processed for MEP morphology and histology. It is estimated that 0.5 g of the 6 g total biopsy will be sufficient for analysis. Functional outcomes of the denervated muscles that have become reinnervated in the post-operative period will be objectively assessed by physical examination detailing muscle strength, joint range of motion, presence/absence of normal muscle bulk return, and as well as EMG criteria. Assessment includes the two types of polyphasic potentials following axonal degeneration: 1) nascent potentials and 2) motor units formed from terminal collateral sprouting. True axonal regeneration leads to the formation of nascent potentials, which are usually low in amplitude, polyphasic in configuration, and of varying duration. Terminal collateral sprouting always leads to the formation of long-duration polyphasics. Nerve conductions will also be performed to assess for the CMAP amplitude, Motor Unit Number Estimation (MUNE), and return of sensory potentials. Multifactorial statistical analyses will be used to evaluate if the presence of MEPs or morphological features on the pre-operative muscle biopsy correlate with improved functional recovery.

### Identification of Human-Specific Surrogate Molecular Markers of Reinnervation-Competent Human Muscle Following Nerve Injury Using Pre-Operative Muscle Biopsy

Measures. Biopsy samples obtained as described above will be assessed using the state-of-the art proteomic technique of tandem mass tagging to investigate the synaptic proteome. MEP-enriched human samples will be micro-dissected from denervated muscle biopsies at multiple time-points, as will innervated control biopsies. Muscle fibers will be labelled with α-bungarotoxin for 5 minutes to identify the location of the MEP bands before the MEP-enriched portions can be micro-dissected under a fluorescence microscope. These MEP-enriched only samples will be collected to measure the protein-level composition of MEPs from each patient sample according to a previously established method. Proteomics technology will help to definitively decipher the functional molecular targets involved that have been permanently translated and part of the interacting protein network. Proteomic profiles will be generated from each patient. Specifically, the generated peptide masses from tandem mass tagging experiments will be searched against two-unified non-redundant databases for protein identification. A small molecule discovery analysis software for proteomics will be used to process and search the data to accurately quantify and identify proteins that are significantly changing between denervated and control muscle samples. Thousands of reviewed non-redundant entries from a human protein sequence database will be downloaded and search algorithm will be applied. Normalized label-free quantification will be achieved and the generated differentially expressed data will be filtered to show only statistically (ANOVA) significantly regulated proteins (p≤0.05) and a fold change >1.5. Binarization will also be applied, and alternating dilation and erosion mathematical operations will be performed to fill small gaps and separate close objects. Markers identified with proteomics will then be confirmed by measuring protein and mRNA expression levels using western blotting and quantitative real-time polymerase chain reaction, respectively. The MEP proteomic profile from each patient will be correlated with preservation of MEPs and eventual functional recovery or lack thereof to identify potential human-specific surrogate markers likely responsible for MEP preservation and thus reinnervation-competent muscle.

Outcomes. One goal is to develop new prognostic/diagnostic criteria and objective, predictive tools to inform surgical decision-making following a traumatic peripheral nerve injury. The present invention describes two possible tools to guide surgical intervention. The first tool dictates that if the presence of certain morphometric characteristics of MEPs on pre-operative muscle biopsy correlates with functional recovery as a maker of successful nerve transfer, then a nerve transfer procedure is indeed indicated. In light of no published human data with regards to the critical timing for surgical intervention and reinnervation, such outcome would suggest that the presence of certain MEPs on pre-operative biopsy of denervated muscle is an indicator that the critical window for surgical intervention and reinnervation still exists and a nerve transfer is still viable. The second tool holds the potential of screening a positive or negative surrogate marker of MEP preservation and muscle reinnervation-competency before deciding on surgical intervention. The innovation here lies in the development of predictive tools of pre-operative muscle biopsy that can guide surgeons in making objective, data-driven predictions of which candidate nerve transfers are likely to be successful in a patient-specific, time-independent manner along with molecular signatures of reinnervation-competency. If rigorous analysis and evaluation demonstrate the validity of these objective, predictive tools, then the landscape of surgical care and management of patients suffering traumatic nerve injuries will be transformed towards more data-driven clinical decision-making.

To Develop a Model System within the CTSA Framework for Assessing Outcomes from Surgical Interventions to Generate Evidence-Based Diagnostic Guides

Problems. Clinical translational research involving surgical procedures is among the most challenging in biomedical discovery.

Solutions & Rationale. The present invention the study of critical elements of translation in the context of surgical clinical research.

Advances in surgical therapies for MEP injury have been inhibited by the dependence upon translational model that did not accurately reflect human-specific neuroanatomic pathways. Creating safe and effective treatments and diagnostic/prognostic tools to improve human health requires rigorous and successful testing of those interventions in humans. However, a barrier that surgical researchers nationwide face is in the recruitment of sufficient and eligible participants for clinical/surgical trials. This inability to identify and recruit the right number and type of patients often results in slow and costly trials or the premature closing of a study altogether. Perhaps even more disheartening and demoralizing is when insufficient numbers and types of patient participants limit the validity of trial results and ultimately the ability to apply the findings broadly for translation to the general population. A major roadblock to dissemination of clinical research findings has been the lack of data and terminology harmonization. Often unappreciated or even ignored is the importance of ensuring that key clinical terms and concepts are agreed upon in advance of the design of clinical trials.

### Innovation

Considering the species-specific differences, understanding human-specific molecular mechanisms involved in human MEP degradation is key to breakthrough discoveries that will redefine human-specific therapeutic targets. The first step towards human-specific mechanistic discoveries is via the state-of-the art proteomic technique of tandem mass tagging, which will be used to attempt to identify surrogate molecular markers of reinnervation-competent muscle at the MEP at multiple time-points following nerve injury from different patients. Beyond the identification of potential surrogate molecular markers of reinnervation-competent muscle from the bioinformatics and in silico analyses of the patient proteomic profiles at each time point, the human-specific data will ultimately aid in forming of future testable hypotheses about the precise molecular mechanism of human-specific MEP degeneration which will advance translational efforts.

### Clinical Treatment

MEP Preservation Following Nerve Injuries. Both the time course of human MEP degeneration as well as timing of adjuvant therapies to preserve MEPs has not been established. To determine the critical timing for any adjuvant therapies directed at augmented preservation of the MEPs, it is necessary to first understand how human MEP degeneration differs from murine MEP degeneration in both process and timing. As such, the present invention describes histologically defining the morphology and temporal profile of MEP degradation in humans following nerve injury. Two-photon excitation microscopy will be used, which permits superior optical visualization in three-dimensional imaging of thick specimens. The deeper penetration of two-photon excitation compared to standard confocal imaging makes it possible to visualize and assess the three-dimensional spatial arrangement and morphometric properties of MEPs in whole muscle biopsy mounts. Images obtained via two-photon excitation microscopy will allow for three-dimensional reconstructions to allow for precise quantification and analyses of morphometric properties of MEPs. Our preliminary results of SA1 represent the first step towards characterizing post-injury degradation of human MEPs to determine the optimal timing for adjuvant treatments aimed at augmenting preservation of human MEPs.

Predicting Surgical Candidacy for Successful Nerve Transfer. The development of an objective tool to assess surgical candidacy for successful nerve transfer is imperative. The significant issue in surgical practice is that the decision to undertake a nerve transfer is currently made clinically, with little guidance from objective data and evidence-based research applicable to humans. The current understanding of neurologic injury and regenerative outcomes is based solely on clinical observational studies which suggest that surgical interventions which take place more than 6 months after nerve injury rarely produce meaningful recovery. Moreover, there is consensus, based on murine studies that a critical time window for nerve repair exists, beyond which destabilization of the MEP limits reinnervation by the slowly regenerating axon in murine model. Although this current understanding may imply that post-synaptic MEP degradation has already occurred by that 6-month time-point, preliminary data suggests persistence of MEPs beyond even 5 years of muscle denervation. The preliminary results described herein suggest that a pre-operative muscle biopsy may be an objective, predictive tool that to aid in the surgical decision-making process by providing direct visualization of the MEP, including its innervation status and morphometric properties. The rationale is that if human MEPs persist and retain their structures even after the previously postulated 6-month therapeutic window for operative intervention, then a nerve transfer may indeed be indicated. This proposed, novel tool of pre-operative muscle biopsy can guide surgeons in making data-driven predictions of which candidate nerve transfers are likely to be successful in a patient-specific, time-independent manner along with molecular signatures of reinnervation-receptivity. Results of this study would represent a paradigm shift from one in which surgical decision-making is based not on time from injury, but rather one driven by quantitative data, such as indication of viable MEPs on muscle biopsy as well as presence of surrogate markers of reinnervation-competency.

The studies of the present invention will transform the medical and surgical care of patients suffering traumatic nerve injuries by defining the critical timing for adjuvant therapy and surgical intervention as well as screening for human-specific surrogate markers of reinnervation-competency to guide surgical-decision making and improve functional outcomes.

### Example 2

The following is a non-limiting example of the present invention. It is to be understood that said example is not intended to limit the present invention in any way. Equivalents or substitutes are within the scope of the present invention.

Various embodiments include a method of treating nerve injury in an individual, comprising providing a composition comprising one or more of the following: agrin, an inhibitor of the matrix metalloproteinase 3 (MMP3) signaling pathway, an inhibitor of the WNT signaling pathway, and an inhibitor of the beta-catenin signaling pathway, and administering a therapeutically effective dosage of the composition to the individual. In another embodiment, the composition is administered in conjunction with surgical treatment. In another embodiment, the individual is a human. In another embodiment, the inhibitor of the MMP3 signaling pathway is an inhibitor of MMP3. In another embodiment, the inhibitor of the WNT signaling pathway is an inhibitor of Wnt3a. In another embodiment, the nerve injury is treated by preserving the neuromuscular junction (NMJ). In another embodiment, administering the composition prevents degradation of the motor end plate after prolonged denervation. In another embodiment, the composition is administered prior to nerve injury surgery. In another embodiment, the composition is administered post nerve injury surgery. In another embodiment, the composition is administered intravenously. In another embodiment, the inhibitor of the MMP3 signaling pathway is selected from the following: minocycline, MMP Inhibitor II, MMP Inhibitor V, CP 471474, MMP-3 Inhibitor I, MMP-3 Inhibitor II, MMP-3 Inhibitor III, MMP-3 Inhibitor IV, actinonin, MMP-3 Inhibitor V, MMP-3 Inhibitor VIII, MMP-13 Inhibitor I, NNGH, PD166793, UK 370106, UK 356618. In another embodiment, the inhibitor of the MMP3 signaling pathway is an MMP3 siRNA molecule. In another embodiment, the inhibitor of the WNT signaling pathway is a Wnt3a siRNA molecule. In another embodiment, the inhibitor of the WNT signaling pathway is an inhibitor of the armadillo protein β-catenin. In another embodiment, the inhibitor of the WNT signaling pathway is an inhibitor of one or more of the following: beta-catenin destruction complex, WNT/Beta-catenin signalsome, cadherin junctions, and hypoxi sensing system Hif-1alpha (hypoxia induced factor 1beta). In another embodiment, the inhibitor of the WNT signaling pathway is one or more of the following: XAV939, IWR1, IWP-1, IWP-2, JW74, JW55, okadaic acid, tautomycein, 2-[4-(4-fluoro-phenyl)piperazin-1-yl]-6-2 methylpyrimidin-4(3H)-one, niclosamide, cambinol, sulindac, filipin, bosutinib, imatinib, ethacrynic acid, PKF118-744, BC21, and Rp-8-Br-cAMP.

Other embodiments include a composition comprising a therapeutically effective dosage of a composition comprising one or more of the following: agrin, an inhibitor of the matrix 5 metalloproteinase 3 (MMP3) signaling pathway, an inhibitor of the WNT signaling pathway, and an inhibitor of the beta-catenin signaling pathway, and a pharmaceutically acceptable carrier. In another embodiment, the inhibitor of the MMP3 signaling pathway is an inhibitor of MMP3. In another embodiment, the inhibitor of MMP3 is an MMP3 antibody. In another embodiment, the inhibitor of MMP3 is selected from the following: minocycline, MMP Inhibitor II, MMP 10 Inhibitor V, CP 471474, MMP-3 Inhibitor I, MMP-3 Inhibitor II, MMP-3 Inhibitor III, MMP-3 Inhibitor IV, actinonin, MMP-3 Inhibitor V, MMP-3 Inhibitor VIII, MMP-13 Inhibitor I, NNGH, PD166793, UK 370106, UK 356618. In another embodiment, the inhibitor of the WNT signaling pathway is an inhibitor of Wnt3a. In another embodiment, the inhibitor of Wnt3a is a Wnt3a antibody. In another embodiment, the inhibitor of MMP3 signaling pathway is selected 15 from the following: XAV939, IWR1, IWP-1, IWP-2, JW74, JW55, okadaic acid, tautomycein, 2-[4-(4-fluoro-phenyl)piperazin-1-yl]-6-methylpyrimidin-4(3H)-one, niclosamide, cambinol, sulindac, filipin, bosutinib, imatinib, ethacrynic acid, PKF118-744, BC21, and Rp-8-Br-cAMP. Other embodiments include a method of preventing nerve injury in an individual, comprising providing a composition comprising one or more of the following: agrin, an 20 inhibitor of the matrix metalloproteinase 3 (MMP3) signaling pathway, an inhibitor of the WNT signaling pathway, and an inhibitor of the beta-catenin signaling pathway, and administering a therapeutically effective dosage of the composition to the individual prior to nerve injury. In another embodiment, the composition is administered intravenously. Various other embodiments include methods of preserving the motor end plate after nerve injury in a subject, comprising providing a composition comprising MMP3 pathway specific siRNA, WNT pathway specific siRNA, and beta-catenin pathway specific siRNA; and transfecting one or more cells of the subject with the composition.

Embodiments of the present invention can be freely combined with each other if they are not mutually exclusive.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A method of treating denervated muscle tissue in a patient in need thereof, said method comprising:
a. performing a pre-operative muscle biopsy on the denervated muscle tissue;
b. making visible motor end plates (MEPs) in neuromuscular junctions (NMJs) in the biopsy; and
c. performing a nerve transfer if (i) the MEPs shown in the biopsy persist and (ii) the MEPs shown in the biopsy retain their structures and exhibit certain morphometric properties.
wherein the nerve transfer helps regain neuromuscular function of the denervated muscle tissue.

2. The method of claim 1, wherein (c) further comprises determining an innervation status of the MEPs in the biopsy.

3. The method of claim 1, wherein the morphometric properties of the MEPs are mature pretzel appearance and/or plaque like phenotype.

4. The method of claim 1, wherein (c) further comprises determining viability of the MEPs.

5. The method of claim 1, wherein the method is performed at least 6 months after injury to the patient.

6. The method of claim 1, wherein two-photon microscopy is performed on the biopsy.

7. The method of claim 1 further comprising detecting at least one surrogate marker of reinnervation-competent muscle.

8. The method of claim 1, wherein the denervated muscle tissue is caused by traumatic injury.

9. The method of claim 1, wherein the nerve transfer is a partial radial nerve to axillary transfer.

10. The method of claim 1, wherein the method is for predicting spontaneous neuromuscular recovery.

11. The method of claim 1, further comprising administering a therapeutic agent to the denervated muscle tissue.
